(19) United States
(12) Patent Application Publication
(10) Pub. No.: US 2023/0030888 A1
(43) Pub. Date: Feb. 2, 2023

(54) RUNOFF ESTIMATING METHOD AND DEVICE FOR UNGAUGED REGION, COMPUTER DEVICE, AND STORAGE MEDIUM

(71) Applicant: TSINGHUA UNIVERSITY, Beijing (CN)

(72) Inventors: DI LONG, Beijing (CN); XUE-YING LI, Beijing (CN)

(21) Appl. No.: 17/697,915

(22) Filed: Mar. 17, 2022

(30) Foreign Application Priority Data
Jan. 18, 2021 (CN) 202110065530.4

(51) Int. Cl. G01N 33/24; G01W 1/00

(52) U.S. Cl. CPC G01N 33/246; G01W 1/00

(57) ABSTRACT

Disclosed are a runoff estimating method and a runoff estimating device for an ungauged region. The method includes: acquiring driving data, the driving data comprising monthly-scale precipitation, monthly-scale potential evapotranspiration, and soil water volume content of a basin within a preset period; establishing an objective function of monthly-scale water quantity balance change of the basin, and determining a parameter to be calculated in the objective function; initializing the parameter to obtain an initial value of the parameter; inputting the initial value of the parameter and the driving data into the objective function to obtain an initial function value of the objective function; performing an iterative computation on the parameter based on the initial value of the parameter and the initial function value to obtain an optimized value of the parameter, and inputting the optimized value of the parameter into the objective function to obtain an objective function value.

### CROSS-REFERENCE TO RELATED APPLICATION

This application claims all benefits accruing under 35 U.S.C. § 119 from China Patent Application No. 202110065530.4, filed on Jan. 18, 2021 in the China National Intellectual Property Administration, the content of which is hereby incorporated by reference.

### TECHNICAL FIELD

The application relates to the technical field of hydrology and water resources management, particularly to a runoff estimating method and a runoff estimating device for an ungauged region, a computer device, and storage medium.

### BACKGROUND

The runoff of a river is an important variable in the hydrological cycle, and it is also the basic data for a regional water resources evaluation, an ecological protection and a climate change analysis. The relevant runoff detection data mainly come from hydrological monitoring stations. The accuracy of the runoff detection data is high, but the distribution of global stations is extremely uneven, and observation periods of most stations are short, and restricted by terrains and observation costs, many ungauged regions lack of reliable runoff data.

In current researches, the method of building a hydrological model is usually used to simulate the runoff of a basin. However, the related method depends heavily on measured runoff data to calibrate model parameters, and the application of the method is limited in the ungauged region.

At present, the model parameters of the basin are calibrated by using measured runoff data of an adjacent basin and are transplanted to the ungauged area. However, due to the uniqueness of different basins, the simulation accuracy is not high.

### SUMMARY

Based on this, and in view of the above technical problems, it is necessary to provide a runoff estimating method and a runoff estimating device for an unaged region, a computer device, and storage medium, which can improve the accuracy of runoff estimation for the unaged region.

In one aspect, a runoff estimating method for an ungauged region is provided, and the method includes:
acquiring driving data, the driving data comprising monthly-scale precipitation, monthly-scale potential evapotranspiration, and monthly-scale soil water volume content of a basin within a preset period,
establishing an objective function of monthly-scale water quantity balance change of the basin, and determining a parameter to be calculated in the objective function,
initializing the parameter to obtain an initial value of the parameter,
inputting the initial value of the parameter and the driving data into the objective function to obtain an initial function value of the objective function,
performing an iterative computation on the parameter based on the initial value of the parameter and the initial function value to obtain an optimized value of the parameter, and inputting the optimized value of the parameter into the objective function to obtain an objective function value, and
when the objective function value meets conditions for an end of the iterative computation, estimating monthly-scale runoff of the basin within the preset period according to the optimized value of the parameter.

In an embodiment, the performing an iterative computation on the parameter to obtain the optimized value of the parameter comprises:
calculating a gradient function of the objective function to the parameter;
calculating a gradient value of the gradient function under a previous-cycle optimized value of the parameter; and
calculating a current-cycle parameter value according to the gradient value to obtain a current-cycle optimized value of the parameter.

In an embodiment, the calculating the current-cycle parameter value according to the gradient value to obtain the current-cycle optimized value of the parameter comprises:
performing the iterative computation on the parameter along a negative direction of the gradient according to the previous-cycle optimized value of the parameter, the gradient value, and a preset optimization accuracy, to obtain the current-cycle optimized value of the parameter.

In an embodiment, a plurality of parameters to be calculated are configured and form a parameter space.

The calculating the gradient function of the objective function to the parameter comprises: calculating gradient functions of the objective function to the plurality of parameters in the parameter space, respectively.

The calculating the gradient value of the gradient function under the previous-cycle optimized value of the parameter comprises: calculating gradient values of the gradient function under previous-cycle optimized values of the plurality of parameters, respectively.

The performing the iterative computation on the parameter along the negative direction of the gradient according to the previous-cycle optimized value of the parameter, the gradient value, and the preset optimization accuracy, to obtain the current-cycle optimized value of the parameter, comprises: performing the iterative computations on the plurality of parameters along the negative direction of the gradient, according to the previous-cycle optimized values of the plurality of parameters, corresponding gradient values, and the optimization accuracy, to obtain the current-cycle optimized values of the plurality of parameters, and the current-cycle optimized values of the plurality of parameters constitute the current-cycle optimized parameter space.

The inputting the optimized value of the parameter into the objective function to obtain the objective function value comprises: inputting the optimized values of the plurality of parameters into the objective function to obtain an objective function value.

In an embodiment, the establishing the objective function of monthly-scale water quantity balance change of the basin comprises: converting a water flux change in the water quantity balance model to obtain a first soil water quantity change parameter,

obtaining a second soil water quantity change parameter according to a water quantity state change in the water quantity balance model, and

establishing the objective function of the monthly-scale water quantity balance change of the basin according to the first soil water quantity change parameter and the second soil water quantity change parameter.

In an embodiment, a building process of the water quantity balance model comprises building the water quantity balance model according to a relationship between an input water flux function, an output water flux function, and the water quantity state within the preset period.

In an embodiment, the parameter comprises a first parameter, a second parameter, a third parameter, a fourth parameter, a fifth parameter, and a sixth parameter.

An establishing process of the output water flux function comprises: obtaining the output water flux function by combining an actual evapotranspiration function, a soil deep percolation function, and a basin runoff function.

The actual evapotranspiration function comprises a functional relationship between the monthly-scale potential evapotranspiration of the basin, the soil water volume content, the first parameter, and the second parameter; the first parameter represents a systematic deviation of an estimated potential evapotranspiration, and the second parameter is configured to determine a curve shape of the actual evapotranspiration function.

The soil deep percolation function comprises a functional relationship between the soil water volume content, the third parameter, and the fourth parameter; the third parameter represents a maximum value of the deep percolation, and the fourth parameter represents a water loss rate of a water-containing soil layer.

The basin runoff function comprises a functional relationship between the input water flux function, the soil water volume content, and the fifth parameter; the fifth parameter represents a runoff-forming rate of precipitation in the input water flux function.

The method further comprises determining the sixth parameter according to the water quantity balance model; the sixth parameter represents a thickness of a researched soil layer of the basin.

In another aspect, a runoff estimating device for an ungauged region is provided and includes:
an acquiring module, configured to acquire driving data, the driving data comprising monthly-scale precipitation, monthly-scale potential evapotranspiration, and soil water volume content of a basin within a preset period,
an establishing module, configured to establish an objective function of a monthly-scale water quantity balance change of the basin, and to determine a parameter to be calculated in the objective function,
an initializing module, configured to initialize the parameter to obtain an initial value of the parameter, and to input the initial value of the parameter and the driving data into the objective function to obtain an initial function value of the objective function,
an optimizing module, configured to perform an iterative computation on the parameter based on the initial value of the parameter and the initial function value, to obtain an optimized value of the parameter, and to input the optimized value of the parameter into the objective function to obtain an objective function value, and
a runoff calculating module, configured to, when the objective function value meets conditions for an end of the iterative computation, estimate a monthly-scale runoff of the basin within the preset period according to the optimized value of the parameter.

In another aspect, a computer device is provided, and includes a memory and a processor, a computer program being stored on the memory. When performing the computer program, the processor executes steps below:
acquiring driving data, the driving data comprising monthly-scale precipitation, monthly-scale potential evapotranspiration, and soil water volume content of a basin within a preset period,
establishing an objective function of monthly-scale water quantity balance change of the basin, and determining a parameter to be calculated in the objective function,
initializing the parameter to obtain an initial value of the parameter,
inputting the initial value of the parameter and the driving data into the objective function to obtain an initial function value of the objective function,
performing an iterative computation on the parameter based on the initial value of the parameter and the initial function value to obtain an optimized value of the parameter, and inputting the optimized value of the parameter into the objective function to obtain an objective function value, and
when the objective function value meets preset conditions for an end of the iterative computation, estimating monthly-scale runoff of the basin within the preset period according to the optimized value of the parameter.

In another aspect, a non-transitory computer readable medium is provided. A computer program is stored on the non-transitory computer readable medium. When executed by a processor, the computer program executes following steps:
acquiring driving data, the driving data comprising monthly-scale precipitation, monthly-scale potential evapotranspiration, and soil water volume content of a basin within a preset period,
establishing an objective function of monthly-scale water quantity balance change of the basin, and determining a parameter to be calculated in the objective function,
initializing the parameter to obtain an initial value of the parameter,
inputting the initial value of the parameter and the driving data into the objective function to obtain an initial function value of the objective function,
performing an iterative computation on the parameter based on the initial value of the parameter and the initial function value to obtain an optimized value of the parameter, and inputting the optimized value of the parameter into the objective function to obtain an objective function value, and
when the objective function value meets preset conditions for an end of the iterative computation, estimating monthly-scale runoff of the basin within the preset period according to the optimized value of the parameter.

According to the runoff estimating method and the runoff estimating device for the ungauged region, the computer device, and the non-transitory computer readable medium above, firstly, the driving data are acquired, the driving data includes the monthly-scale precipitation, the monthly-scale potential evapotranspiration, and the soil water volume content of the basin within the preset period. Then the objective function of monthly-scale water quantity balance change of the basin is established, and a parameter to be calculated in the objective function is determined, the parameter is initialized to obtain an initial value of the parameter, and the initial value of the parameter and the driving data are input into the objective function to obtain the initial function value of the objective function, and an iterative computation is performed on the parameter based on the initial value of the parameter and the initial function value. Finally, the monthly-scale runoff of the basin within the preset period is estimated according to the optimized value of the parameter. The method, independent of any measured runoff data, may estimate the runoff for the ungauged region by inputting fewer driving data, and the estimation accuracy is high.

### BRIEF DESCRIPTION OF THE DRAWINGS

FIG. 1 is a schematic flowchart of a runoff estimating method for an ungauged region according to an embodiment;

FIG. 2 is a schematic diagram showing a monthly-scale water quantity balance model of a basin according to an embodiment;

FIG. 3 is an overall frame view showing the runoff estimating method for the ungauged region according to an embodiment;

FIG. 4 is a diagram showing tested objects of the runoff estimating method for the ungauged region according to an embodiment;

FIG. 5 is a diagram showing the test results of the runoff estimating method for the ungauged region according to an embodiment;

FIG. 6 is a structural block view showing a runoff estimating device for an ungauged region according to an embodiment;

FIG. 7 is a view showing an internal structure of a computer device according to an embodiment.

### DETAILED DESCRIPTION OF THE EMBODIMENTS

In order to make the objectives, technical solutions and advantages of the present disclosure clearer and better understood, the application will be further described in detail below with reference to the accompanying drawings and embodiments. It should be understood that the specific embodiments described herein are merely illustration of the application, but not intended to limit the application.

The runoff of a river is an important variable in the hydrological cycle, and it is also the basic data for a regional water resources evaluation, an ecological protection and a climate change analysis. According to the hydrological process formed by the runoff, the total runoff includes surface runoff, subsurface runoff, groundwater runoff. The relevant runoff detection data mainly come from hydrological monitoring stations. The accuracy of the runoff detection data is high, but the distribution of global stations is extremely uneven, and observation periods of most stations are short, and restricted by terrains and observation costs, many ungauged regions lack of reliable runoff data. In hydrological research, the method of building a hydrological model is usually used to simulate the runoff of the basin. However, the related method depends heavily on the measured runoff data to rate model parameters, and the application of the method is limited in the ungauged region.

The runoff simulation for the ungauged region is one of the difficulties in the hydrology research, and common methods mainly include parameter transplantation methods such as an analogy method, an interpolation method, and a regression method, etc. The analogy method and the interpolation method need to use the measured runoff data of the adjacent basin, and are sensitive to the spatial transform of the geographical locations and topographies between the simulated basin and the reference basin. In the regression method, a regression relationship, between the basin characteristics of the reference basin and the model parameters, is established, and is applied to other basins, but the effect achieved by this method is often limited. Due to the uniqueness of different basins, the parameter transplantation method has great uncertainty.

In recent years, based on the water quantity balance equation, the methods of deriving various quantities such as precipitation, evapotranspiration, runoff, lateral flow and infiltration and irrigation amount from the basin water quantity state (water quantity in the soil), have been developed gradually. For example, Brocca et al. estimates the precipitation by using the measured soil water quantity changes, Akbar et al. estimates the grid-scale evapotranspiration and discharge by using the soil water quantity information observed by the remote sensing satellite SMAP, and Filippucci et al. calculates the irrigation amount in the irrigation area by using the measured soil water quantity and the measured precipitation information. These methods have clear physical principles, and compared with the traditional hydrological model, the input driving data are less, and the calculation methods thereof mostly adopt the idea of mathematical optimization, which may overcome the limitation that the measured data are required by the hydrological model to rate the parameters. However, at present, such researches are usually carried out at the stations or at the grid scale, and it is assumed that the runoff may be negligible. For example, Brocca et al. and Akbar et al. assume that all precipitation may infiltrate and will not form surface runoff. Since excess water runoff is not anticipated in the optimal irrigation, Filippucci et al. assumes that surface runoff is negligible in calculating the irrigation volume. These assumptions may be applied to a smaller scale and a specific situation, but at the basic scale, ignoring the runoff will result in an unclosed water quantity balance, thus causing a relatively large error. In conclusion, such research methods have not been applied to the estimation of the basin runoff yet, and the potential of these methods in the runoff process analysis, in the hydrological cycle research and in the water resources management needs to be further developed.

In order to obtain the runoff sequence in the ungauged region, based on the monthly-scale water quantity balance model of the basin and by using the optimal calculation idea for high-dimensional parameter space, the present application provides a monthly-scale runoff estimating method independent of any measured runoff data, and provides a reference for measuring runoff of the ungauged region. It should be noted that, the ungauged region in the present application refers to a researched basin, which has no measured runoff data but a few other observed data, such as the monthly-scale precipitation of the basin, etc.. Taking these observed data as a reference, it is possible to speed up the convergence rate of the objective function of this application, and even if there is no observed data of the researched basin, the final calculated runoff will not be affected. In the present application, the runoff of the ungauged region is estimated by obtaining driving data from the global reanalysis dataset of ERA5. The method has been successfully applied to the river source areas in the Qinghai-Tibet Plateau, including the Yarlung Zangbo River (taking Nuxia Station as the basin outlet), the Nujiang (taking Jiayuqiao Station as the basin outlet), the Lancang River (taking Qamdo Station as the basin outlet), the Yangtze River (taking Zhimenda Station as the basin outlet), and the Yellow River (taking Tangnaihai Station as the basin outlet), and the method may provide a reference for the runoff estimation of other ungauged basins in the world.

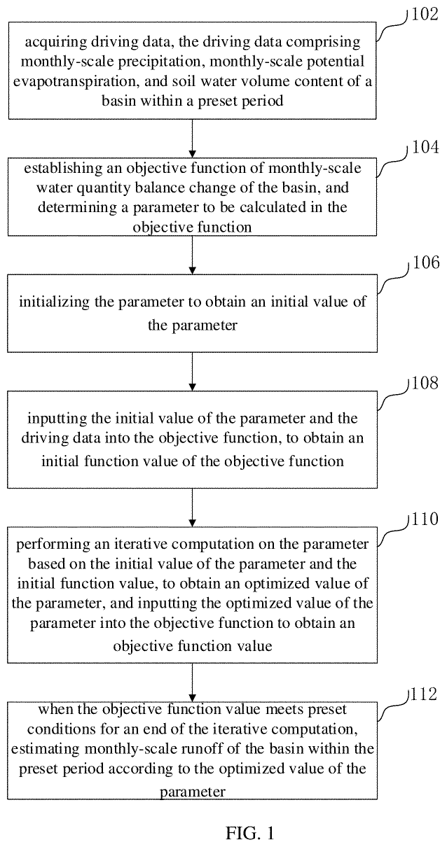

In an embodiment, as shown in FIG. 1, a runoff estimating method for an ungauged region is provided. This embodiment is illustrated by applying the method to a terminal. It should be understood that the method may also be applied to a server, and may be applied to a system including a terminal and a server, and be realized through an interaction of the terminal and the server. In this embodiment, the method includes the following steps.

At Step 102, driving data are acquired, and the driving data includes monthly-scale precipitation, potential evapotranspiration, and soil water volume content of a basin within a preset period.

The driving data are necessarily configured to solve an objective function of a monthly-scale water quantity balance change of the basin, so as to estimate the monthly-scale runoff of the basin. The preset period may be determined according to requirements of the solution and not limited in this embodiment of the application. For example, in order to estimate the runoff from January to December 2019, the preset period is from January to December 2019, and the driving data corresponding to the period are obtained.

Specifically, the driving data within the preset period are obtained from the global reanalysis dataset of ERA5. The driving data in this embodiment of the present application includes the monthly-scale precipitation, the potential evapotranspiration and the soil water volume content of the basin, etc. ERA5 is the fifth generation European Centre for Medium-Range Weather Forecasts (ECMWF) atmospheric reanalysis of the global climate. ERA5 provides hourly estimates of atmospheric, land and oceanic climate variables. The data cover the Earth on a 30 km grid and resolve the atmosphere using 137 levels. The dataset of ERA5 is in netcdf format, and may be read by means of the “ncread” function in the Matlab program.

At Step 104, an objective function of monthly-scale water quantity balance change of the basin is established, and a parameter to be calculated in the objective function is determined.

Specifically, the soil water quantity change relationship of a researched basin is obtained by two calculation methods, and then the objective function to be optimized is established according to the root-mean-square error between the soil water quantity changes calculated by the two methods, and the parameter in the objective function is determined to constitute the parameter space. The objective function may be established according to the root-mean-square error between a soil water quantity change, which is calculated according to the input water flux and the output water flux, and another soil water quantity change, which is calculated according to the monthly-scale water quantity state of the researched basin. The unknown parameter in the objective function is configured to be the parameter to be calculated.

At Step 106: the parameter is initialized to obtain an initial value of the parameter.

Specifically, in order to solve the objective function, it is necessary to assign an initial value to the parameter, that is, to assign an initial value to the parameter space to obtain the initial value of the parameter space. Theoretically, the initial value may be assigned arbitrarily, but in an actual solution, different initial values will affect the convergence rate of the optimization curve of the objective function. In order to avoid invalid operations, the initial value is assigned with reference to the actual situations of the researched basin or the adjacent basin and by combining the physical significance of different parameters. The relatively reasonable initial value may make the solution process converge faster, but it will not affect the final optimization results. For example, assuming that a represents a systematic deviation of the potential evapotranspiration estimated by ERA5, a difference between the potential evapotranspiration obtained from ERA5 and the evapotranspiration in the observation record may be assigned to a as the initial value.

It should be noted that the observation record may help determine the initial value of the parameter, so that the initial state of the objective function of the researched basin is closer to the real state, so as to speed up the convergence process of the objective function, but not to affect the final calculated runoff. Therefore, whether there are observation data of other variables in the researched basin is not required in the embodiment of the present application.

At step 108, the initial value of the parameter to be calculated and the driving data are input into the objective function, to obtain an initial function value of the objective function.

Specifically, the initial value of the above parameter space and the obtained driving data are input into the objective function together for calculation, and the initial function value of the objective function is obtained. The initial function value is compared with a subsequent optimized objective function value, to determine whether the conditions for an end of an iterative computation are met.

At Step 110, an iterative computation is performed on the parameter to be calculated based on the initial value of the parameter and the initial function value, to obtain an optimized value of the parameter, and the optimized value of the parameter are input into the objective function to obtain an objective function value.

Specifically, the optimization method in the mathematical analysis may be used to iteratively optimize the parameter to obtain the optimized value of the parameter, that is, the optimized value of the parameter space. For example, the conjugate gradient method may be used to optimize and solve the parameter in the objective function, and any other optimization method, which is not limited in the embodiment, may also be used to solve the problem. Finally, the optimized value of the parameter are input into the objective function, to obtain the objective function value.

At Step 112, when the objective function value meets preset conditions for the end of the iterative computation, the monthly-scale runoff of the basin within the preset period is estimated according to the optimized value of the parameter.

Specifically, the preset conditions for the end of the iterative computation are requirements of the solution accuracy of the objective function. In the present application, an optimization threshold is set to 1‰ of the magnitude of the soil water quantity change, and the preset conditions for the end are that: if the difference between the objective function values of two optimizations (that is, one objective function value obtained from a current-cycle optimization and another objective function value obtained from the previous-cycle optimization) is less than the optimization threshold, the iteration ends; if the difference between the two optimized objective function values is greater than or equal to the optimization threshold, the next-cycle iterative computation will continue. Then the monthly-scale runoff of the basin within the preset period is calculated according to the optimized value of the parameter in last cycle.

For example,

$$\overline{\vert \frac{d θ}{dt} \vert}$$

denotes the magnitude of the soil water quantity change, Jᵢ₊₁ denotes the objective function value obtained in the current-cycle optimization, and Jᵢ denotes the objective function value obtained in the previous-cycle optimization. If the difference between the two optimized objective function values is less than the optimization threshold, the optimal solution is obtained. That is, when

$$\vert {J}_{i + 1} - {J}_{i} \vert < \overline{\vert \frac{d θ}{dt} \vert} \cdot 0.001$$

is satisfied, the optimization process ends. Finally, the monthly-scale runoff of the basin is solved according to the parameter space Xᵢ₊₁ of the current-cycle optimization and the runoff function. In the inequality,

$$\overline{\vert \frac{d θ}{dt} \vert}$$

represents a mean value of the absolute value of the water quantity changes of all samples involved in the solution.

In the runoff estimating method for the ungauged region above, the driving data are obtained. The driving data include the monthly-scale precipitation, the potential evapotranspiration, and the soil water volume content of the basin within the preset period. Then, the objective function of the monthly-scale water quantity balance change of the basin is established, and the parameter to be calculated in the objective function is determined. Then the parameter is initialized, and the initial value of the parameter and the driving data are input into the objective function to obtain the initial function value of the objective function, and the iterative computation is performed on the parameter based on the initial value of the parameter and the initial function value. Finally, the monthly-scale runoff of the basin within the preset period is estimated according to the optimized value of the parameter. In the method, no measured runoff data are required to estimate the runoff in the ungauged region, and the estimation accuracy is high.

In an embodiment, performing the iterative computation on the parameter to obtain the optimized value of the parameter includes following steps.

A gradient function of the objective function to the parameter is calculated.

A gradient value of the gradient function under the previous-cycle optimized value of the parameter is calculated.

A current-cycle parameter value is calculated according to the gradient value, to obtain the current-cycle optimized value of the parameter.

Specifically, the gradient function represents the gradient of the objective function to the parameter, and may be a derivative of the objective function to the parameter. For example, the gradient of the objective function to the parameter can be obtained by means of the “diff” function in the Matlab program. For example, J denotes the objective function, xᵢ₊₁ denotes the parameter in the current cycle, xᵢ denotes the parameter in the previous cycle, and

$$\frac{\partial J}{\partial {x}_{i}}$$

denotes the gradient of the objective function to the parameter.

Then the gradient value of the objective function to the parameter under the previous-cycle optimized value of the parameter is calculated. Specifically, the parameter xᵢ in the previous cycle is substituted into the gradient function, and the gradient value of the parameter under the condition of xᵢ may be obtained.

Finally, according to the parameter xᵢ and the corresponding gradient value above, the current-cycle optimized value of the parameter xᵢ₊₁ is obtained. The current-cycle optimized value of the parameter is substituted in the objective function for calculation, to obtain the current-cycle objective function value Jᵢ₊₁.

In an embodiment, calculating a current-cycle parameter value according to the gradient value to obtain the current-cycle optimized value of the parameter incudes following steps.

According to the previous-cycle optimized value of the parameter, the gradient value, and a preset optimization accuracy, the iterative computation is performed on the parameter along the negative direction of the gradient, to obtain the current-cycle optimized value of the parameter.

Specifically, according to the previous-cycle optimized value of the parameter, the gradient value, and the preset optimization accuracy, the parameter is made to move in the negative direction of the gradient. In a high-dimensional convex space having an optimal solution (having an extreme value), the opposite direction of the gradient of the parameter space is the fastest direction close to the extreme value, so during the iterative computation for the parameter, if the parameter moves in the opposite direction of the gradient every time, namely

$${x}_{i + 1} = {x}_{i} - α \frac{\partial J}{\partial {x}_{i}} ,$$

the parameter may be approximate to the optimal solution that is, the optimized value of the parameter. Where a represents the optimization accuracy. The optimization accuracy may be given arbitrarily, and it is set to 5 in the embodiment of the application. The optimization accuracy may also be set to any other value according to actual needs, and a reasonable value needs to be given according to the requirements of computation and solution. The smaller α, the higher the accuracy, but the higher the computation cost. In practice, α generally ranges from 1 to 10.

In an embodiment, a plurality of parameters are configured and form a parameter space together.

Calculating a gradient function of the objective function to the parameter, includes a step of calculating gradient functions of the objective function to the parameters, respectively.

Calculating a gradient value of the gradient function under the previous-cycle optimized value of the parameter includes calculating gradient values of the gradient function under corresponding previous-cycle optimized values of the parameters, respectively.

According to the previous-cycle optimized value of the parameter, the gradient value, and the optimization accuracy, performing the iterative computation on the parameter along the negative direction of the gradient, to obtain the current-cycle optimized value of the parameter, includes following steps.

According to the previous-cycle optimized values of the parameters, the corresponding gradient values, and the optimization accuracy, the iterative computations are performed on the parameters along the negative direction of the gradient, to obtain the current-cycle optimized values of the parameters, and the optimized values of the parameters constitute the current-cycle optimized parameter space.

Specifically, the objective function contains a plurality of parameters, which together form a parameter space. For example, X denotes the parameter space of the parameters to be calculated, and X₀ denotes an initial value of the parameter space. During the iterative optimization, the parameters need to be calculated and optimized separately, to obtain the optimized values of the parameters separately. Firstly, the gradient functions of the objective function to the parameters in the parameter space are calculated respectively. Then the respective gradient values of the gradient functions of the parameters are calculated according to the previous-cycle optimized values of the parameters. Finally, according to the previous-cycle optimized values of the parameters, the corresponding gradient values, and the optimization accuracy, each of the parameters is made to move in the negative direction of the gradient to obtain one corresponding optimized parameter value. The optimized parameter values together constitute the current-cycle optimized parameter space Xᵢ₊₁, and Xᵢ₊₁=[aᵢ₊₁, bᵢ₊₁, cᵢ₊₁, dᵢ₊₁, fᵢ₊₁, zᵢ₊₁]

In an embodiment, the building process of the water quantity balance model includes following steps.

The water quantity balance model is built according to the relationship between the input water flux function, the output water flux function, and the water quantity state within the preset period.

Specifically, the monthly-scale water quantity balance model of the basin is built according to the relationship between the monthly-scale input water flux, the output water flux and the monthly-scale water quantity state change of the basin. The water quantity balance equation may be expressed as: the soil water quantity change in the researched basin over a period is equal to the difference between the input water flux (precipitation) and the output water flux (including the actual evapotranspiration, the runoff and the deep percolation).

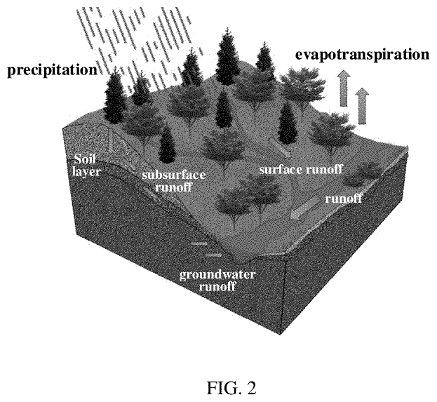

As shown in FIG. 2, taking the soil layer of a certain thickness as the researched object, the input water flux is the precipitation of the basin, and the output water flux includes the actual evapotranspiration, the runoff (including the surface runoff, the subsurface runoff, and the groundwater runoff) and the soil deep percolation. The basin water quantity state change is the water quantity change of the soil of such a thickness in the researched basin. For example, the water quantity balance equation of the monthly scale of the basin may be expressed as:

$$Δ z \frac{d θ}{dt} = P ( t ) - L ( θ ) \qquad ( 1 )$$

In the equation, Δz represents the thickness of the soil layer of the researched basin, is one of the parameters to be calculated in the present application, and is a parameter that satisfies the close of the water quantity balance. Δz not only includes the soil layer thickness corresponding to the measured soil water quantity, but also is related to a dynamics of deeper soil water quantity. θ represents the soil water volume content. P(t) represents the monthly-scale precipitation in mm/mon, that is, the input water flux of the basin. L(θ) represents the output water flux in mm/mon.

In the embodiments of the application, values of θ and P(t) of the monthly scale of the basin may be obtained from the global reanalysis dataset of ERA5. Δz is a free parameter and needs to be given an initial value, and then is continuously updated in the process of parameter iteration, and finally the optimal solution is found. In practice, in order to make the parameter iteration converge faster, the initial value of about 300 cm may be assigned by referring to the soil layer thickness (289 cm) of ERA5.

In an embodiment, establishing an objective function of the monthly-scale water quantity balance change of the basin includes following steps.

A water flux change in the water quantity balance model is converted to obtain a first soil water quantity change parameter.

A second soil water quantity change parameter is obtained according to the water quantity state change in the water quantity balance model.

The objective function of the monthly-scale water quantity balance change of the basin is established according to the first soil water quantity change parameter and the second soil water quantity change parameter.

Specifically, the first soil water quantity change parameter is calculated according to the difference between the input water flux and the output water flux in the water quantity balance model and the thickness of the soil layer of the researched basin. The soil water quantity change calculated according to the water quantity state of the researched basin is taken as the second soil water quantity change parameter. Finally, the objective function of the monthly-scale water quantity balance change of the basin is established according to the root-mean-square error of the first soil water quantity change parameter and the second soil water quantity change parameter. As shown in the following equation (2):

$$J = \sqrt{\frac{1}{N} {\sum }_{t = 1}^{N} {( \frac{d θ}{dt} - ( \frac{P ( t ) - L ( θ , X )}{Δ z} ) )}^{2}} \qquad ( 2 )$$

In the equation, N represents the number of samples, and X represents the parameter space of the parameters to be calculated. If there are six parameters a, b, c, d, r, z, then

$$X = [ a , b , c , d , f , z ] . \frac{P ( t ) - L ( θ , X )}{Δ z}$$

represents the first soil water quantity change parameter, and

$$\frac{d θ}{dt}$$

represents the second soil water quantity change parameter. The number N of samples represents the number of data involved in the solution, which is determined according to the period of the solution. For example, for estimating the runoff from January to December 2019, the number of samples is 12.

In an embodiment, the parameters include a first parameter, a second parameter, a third parameter, a fourth parameter, a fifth parameter, and a sixth parameter.

The establishing process of the output water flux function includes following steps.

The output water flux function is obtained by combining the actual evapotranspiration function, the soil deep percolation function, and the basin runoff function.

The actual evapotranspiration function includes the functional relationship between the monthly-scale potential evapotranspiration of the basin, the soil water volume content, the first parameter, and the second parameter. Where, the first parameter represents a systematic deviation of the estimated potential evapotranspiration, and the second parameter is configured to determine a curve shape of the actual evapotranspiration function.

The soil deep percolation function includes the functional relationship between the soil water volume content, the third parameter and the fourth parameter. Where, the third parameter represents the maximum value of the deep percolation, and the fourth parameter represents a water loss rate of the water-containing soil layer.

The basin runoff function includes the functional relationship between the input water flux function, the soil water volume content, and the fifth parameter. Where, the fifth parameter represents the runoff-forming rate of precipitation in the input water flux function.

The method further includes determining the sixth parameter according to the water quantity balance model. Where, the sixth parameter represents the thickness of the researched soil layer of the basin.

Specifically, the output water flux function is obtained by combining the actual evapotranspiration function, the soil deep percolation function, and the basin runoff function. For example, the component expressions contained in the output water flux function L(θ) are as follows:

L(θ)=ET(θ,a,b)+D(θ,c,d)+R(θ,f) (3)

In the equation, ET(θ, a, b) represents the actual evapotranspiration in mm/mon, and D(θ, c, d) represents the soil deep percolation in mm/mon, and R(θ, f) represents the basin runoff depth in mm/mon, including surface runoff and underground runoff in mm/mon. a, b, c, d, f are all parameters to be calculated for determining the mathematical function form of each of the component expressions, and represent the first parameter, the second parameter, the third parameter, the fourth parameter, and the fifth parameter, respectively.

Each of the component expressions in the equation (3) may be expressed by a math function. The actual evapotranspiration ET may be fitted by a hyperbolic tangent function (equation (4)), and the asymptote thereof is the magnitude of the potential evapotranspiration (the maximum value that characterizes the evapotranspiration of the basin). The key parameters determine the shape of the function curve which characterizes a transformation process of the actual evapotranspiration from being stressed by water quantity to being stressed by energy. The physical meaning of this function is that: when the soil water volume content is zero, the actual evapotranspiration of the basin is zero, and the actual evapotranspiration is stressed by water quantity, and when the soil water volume content is relatively large, that is, when the soil is sufficiently moist, the actual evapotranspiration of the basin is approximate to the potential evapotranspiration, and the function values approach the range of the asymptote, and the actual evapotranspiration is stressed by the energy.

$$ET ( θ , a , b ) = \frac{PET ( t )}{2} \cdot [ 1 + tanh ( 8 \cdot [ \frac{θ}{φ} - sig ( b ) + 0.25 ] ) ] - a \qquad ( 4 )$$

In the equation, PET(t) represents the monthly-scale potential evapotranspiration of the basin in mm/mon, which may be obtained from the global reanalysis dataset of ERA5. The first parameter a represents the systematic deviation of the estimated potential evapotranspiration of ERA5. The function sig(b) of the second parameter b is a sigmoid function, that is

$$sig ( b ) = \frac{1}{1 + {e}^{- b}} .$$

φ represents a soil porosity degree, which may be calculated by the percentage of sand and clay in the soil, that is, φ=(sand×0.395)+(clay×0.482)+(1−sand−clay)×0.451, where, sand and clay represent the mass percentages of the sand and the clay in the total soil layer respectively, which may be obtained from the Global Soil Properties Dataset.

In the initialization phase, the soil porosity degree φ, together with the driving data such as the monthly-scale-scale precipitation of the basic and the potential evapotranspiration and the soil water volume content, and the initial values of the parameters, is input into the objective function, to obtain the initial function value of the objective function.

The deep percolation D includes a soil lateral flow and a vertical deep percolation, and the mathematical function thereof may be expressed as a power function of the soil water volume content:

$$D ( θ , c , d ) = c \cdot {( \frac{θ}{φ} )}^{d} \qquad ( 5 )$$

In the equation (5), the third parameter c represents the maximum deep percolation, and the fourth parameter d may represent the water loss rate of the water-containing soil layer.

The math function of the runoff R may be expressed as a power function introducing the precipitation and the soil water volume content, ant the maximum value of the function is equal to the basin precipitation:

$$R ( θ , f ) = P ( t ) \cdot {( \frac{θ}{φ} )}^{f} \qquad ( 6 )$$

In the equation (6), the fifth parameter f may represent the runoff-forming rate of precipitation, namely, the rate at which the precipitation is transformed into the runoff.

In the water quantity balance model (i.e. the equation (1)), the sixth parameter Δz represents the thickness of the soil layer of the researched basin.

Different from the traditional hydrological model that relies on a large amount of measured surface runoff to calibrate the parameters of the model, the runoff estimating method for the ungauged region provided by the embodiment of the present application does not require any measured runoff data, requires fewer driving data, has concisely generalized equations, has strong applicability, and may be applied to many fields such as water resource utilization and management, hydrological process and climate change research. The application of this method may provide data reference for runoff monitoring for the ungauged region, and may effectively solve the problem that the measured runoff data are necessary for the hydrological model.

In the embodiments of this application, the runoff estimating method for the ungauged region is also tested in five river source areas of the Qinghai-Tibet Plateau, by simulating the monthly-scale runoff of the basin from 2000 to 2017, and by using the runoff observed by the hydrological stations at the basin outlets of the basins of the Brahmaputra (taking Nuxia Station as the basin outlet), the Nujiang (taking Jiayuqiao Station as the basin outlet), the Lancang River (taking Qamdo Station as the basin outlet), the Yangtze River (with Zhimenda Station as basin outlet), the Yellow River (with Tangnaihai Station as basin outlet). The test index of the embodiment of the present application is the Nash efficiency coefficient NSE, and the calculation equation is as follows:

$$NSE = 1 - \frac{{( {Q}_{m} - {Q}_{o} )}^{2}}{{( {Q}_{o} - \overline{{Q}_{o}} )}^{2}} \qquad ( 7 )$$

In the equation, Qₘ and Qₒ represent the calculated runoff and the measured runoff, respectively, Q̅ₒ̅ represents an average value of the measured values, and the theoretical optimal value of this index is 1.

It should be noted that although the five basins above have measured runoff data, during the test for the present application, the measured runoff data are not involved in the calculation, and these basins are regarded as ungauged regions, and the runoff of these ungauged regions is estimated by obtaining the driving data from the global reanalysis dataset of ERA5. Finally, the accuracy of the runoff estimating method for the ungauged region provided in this application is tested by using the measured runoff data of these five basins and the runoff data estimated by the application, to verify the validity of the method.

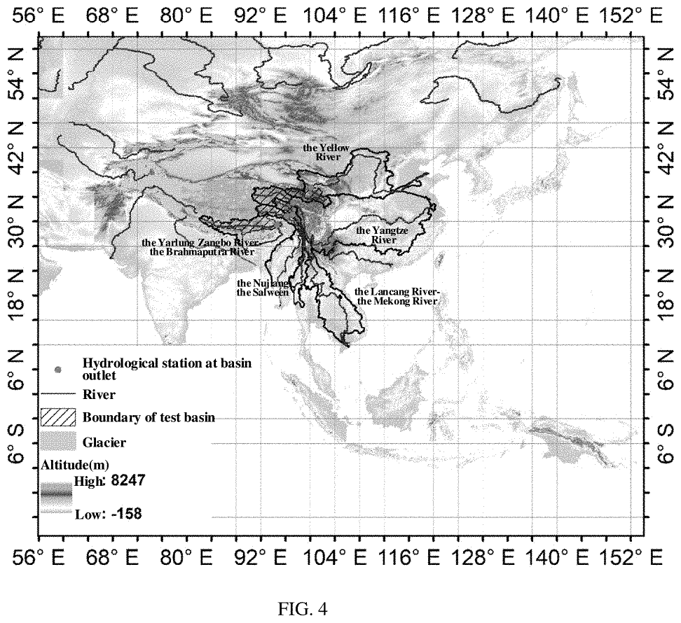

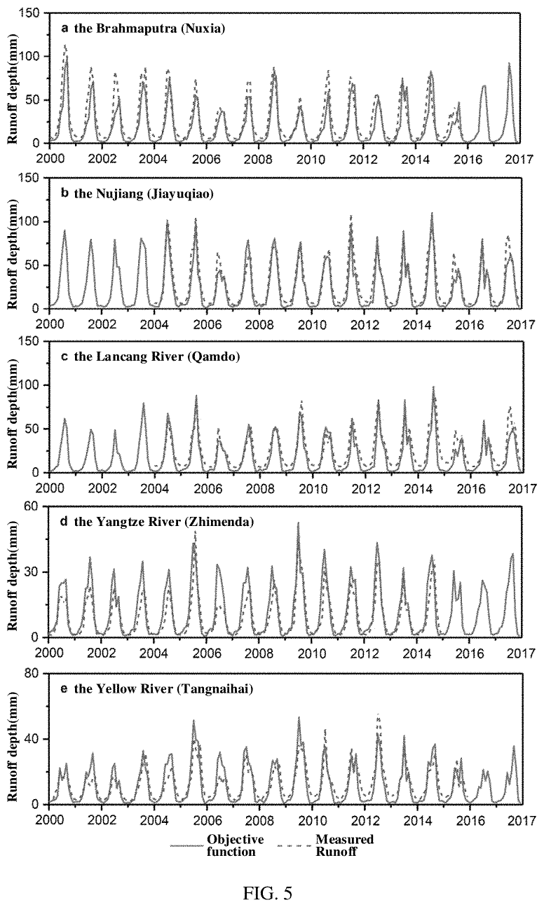

The geographic locations of the researched basin and the observation stations are shown in FIG. 4, and as shown in FIG. 5, the results show that, in the case of completely independent of the measured runoff, compared with the measured runoff, the calculated results of the monthly-scale runoff of the five test basins have the Nash efficiency coefficients (NSE) all greater than 0.6, and the NSE of 0.85 in the Nujiang Basin is the highest. The runoff calculated by the optimization method may more accurately reflect the interannual variation, the peak value and the magnitude. The verification results show that the runoff estimating method for the ungauged region provided by the embodiment of the present application may solve the difficult problem of the runoff simulation for the ungauged region, and has strong adaptability and high accuracy for the ungauged region.

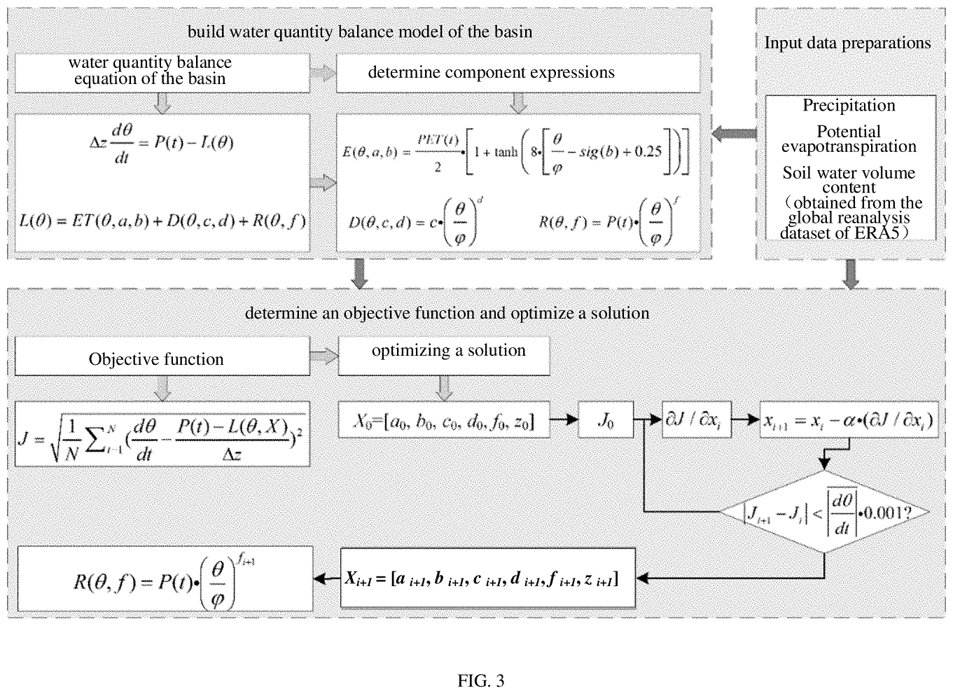

In order to make the technical solutions provided by the embodiments of the present application easily understood, as shown in FIG. 3, the runoff estimating method for the ungauged region provided by the embodiments of the present application is briefly described herein by means of a complete process of estimating the runoff for the ungauged region.

1. The water quantity balance model, namely the Equation (1), is built according to the relationship of the difference between the input water flux function P(t) and the output water flux function L(θ) and the water quantity state change

$$\frac{d θ}{dt}$$

within the preset period.

2. The output water flux function, namely the Equation (3), is obtained according to the actual evapotranspiration function ET(θ, a, b), the soil deep percolation function D(θ, c, d), and the basin runoff function R(θ, f).

The actual evapotranspiration function (i.e., the Equation (4)) includes the functional relationship between the monthly-scale potential evapotranspiration of the basin, the soil water volume content, the first parameter, and the second parameter. Where, the first parameter represents a systematic deviation of the estimated potential evapotranspiration, and the second parameter is configured to determine the curve shape of the actual evapotranspiration function.

The soil deep percolation function (i.e., the Equation (5)) includes the functional relationship between the soil water volume content, the third parameter and the fourth parameter. Where, the third parameter represents the maximum value of the deep percolation, and the fourth parameter represents the water loss rate of the water-containing soil layer.

The basin runoff function (i.e., the Equation (6)) includes the functional relationship between the input water flux function, the soil water volume content, and the fifth parameter. Where, the fifth parameter represents the runoff-forming rate of precipitation in the input water flux function.

The sixth parameter is determined according to the water quantity balance model. The sixth parameter represents the thickness of the researched soil layer of the basin.

3. The water flux change in the water quantity balance model is converted to obtain the first soil water quantity change parameter.

The second soil water quantity change parameter is obtained according to the water quantity state change in the water quantity balance model.

The objective function of the monthly-scale water quantity balance change of the basin, namely the Equation (2), is established according to the first soil water quantity change parameter and the second soil water quantity change parameter.

4. The driving data are obtained. The driving data include the monthly-scale precipitation, the potential evapotranspiration, and the soil water volume content of the basin within the preset period.

5. The parameter is initialized to obtain the initial value of the parameter.

The initial value of the parameter and the driving data are input into the objective function, to obtain the initial function value of the objective function.

6. The iterative computation is performed on the parameters based on the initial values of the parameters and the initial function values. That is, gradient functions of the objective function to the parameters are calculated, respectively, and gradient values of the gradient functions under the previous-cycle optimized values of the parameters are calculated, respectively. According to the previous-cycle optimized values of the parameters, the corresponding gradient values, and the optimization accuracy, the iterative computations are performed, to obtain the corresponding optimized values of the parameters, and the optimized values of the parameters constitute the optimized parameter space. The optimized values of the parameters are input into the objective function to obtain the current-cycle objective function value.

7. When the objective function value meets the preset conditions for the end of the iterative computation, the monthly-scale runoff of the basin is estimated according to the optimized values of the parameters.

8. The accuracy of the estimated monthly-scale runoff of the basin is calculated.

It should be understood that although the steps in the flowcharts of FIGS. 1 and 3 are sequentially displayed according to the arrows, these steps are not necessarily executed in the order indicated by the arrows. Unless explicitly stated herein, the execution of these steps is not strictly limited to the order, and these steps may be performed in other orders. Moreover, at least part of the steps in FIGS. 1 and 3 may include a plurality of steps or a plurality of stages. These steps or stages are not necessarily executed simultaneously, but may be executed at different times. The order in which these steps or stages are performed is also not necessarily in sequence, but may be performed sequentially or alternately with other steps or at least part of the steps or phases within the other steps.

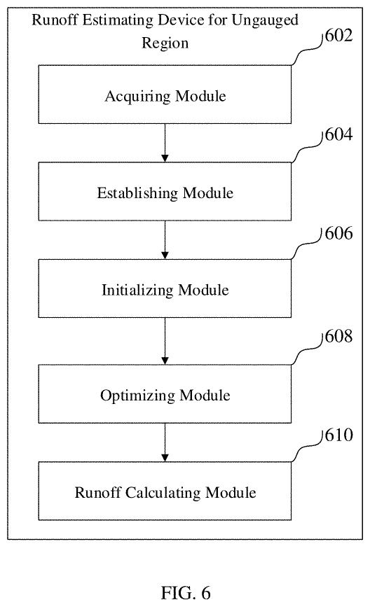

In an embodiment, as shown in FIG. 6, a runoff estimating device for an ungauged region is provided, and includes an acquiring module 602, an establishing module 604, an initializing module 606, an optimizing module 608, and a runoff calculating module 610.

The acquiring module 602 is configured to acquire driving data, and the driving data includes monthly-scale precipitation, potential evapotranspiration and soil water volume content of a basin within a preset period.

The establishing module 604 is configured to establish an objective function of a monthly-scale water quantity balance change of the basin, and to determine a parameter in the objective function.

The initializing module 606 is configured to initialize the parameter to obtain an initial value of the parameter, and to input the initial value of the parameter and the driving data into the objective function to obtain an initial function value of the objective function.

The optimizing module 608 is configured to perform an iterative computation on the parameter based on the initial value of the parameter and the initial function value, to obtain an optimized value of the parameter, and to input the optimized value of the parameter into the objective function to obtain an objective function value.

The runoff calculating module 610 is configured to, when the objective function value meets preset conditions for an end of the iterative computation, estimate a monthly-scale runoff of the basin within the preset period according to the optimized value of the parameter.

In an embodiment, the establishing module 604 is further configured to build a water quantity balance model according to a relationship between an input water flux function, an output water flux function, and a water quantity state within the preset period.

In an embodiment, the establishing module 604 is further configured to convert a water flux change in the water quantity balance model to obtain a first soil water quantity change parameter.

The establishing module 604 is configured to obtain a second soil water quantity change parameter according to the water quantity state change in the water quantity balance model.

The establishing module 604 is configured to establish the objective function of the monthly-scale water quantity balance change of the basin according to the first soil water quantity change parameter and the second soil water quantity change parameter.

In an embodiment, the parameters include a first parameter, a second parameter, a third parameter, a fourth parameter, a fifth parameter, and a sixth parameter.

The establishing module 604 is further configured to obtain an output water flux function by combining an actual evapotranspiration function, a soil deep percolation function, and a basin runoff function.

The actual evapotranspiration function includes the functional relationship between the monthly-scale potential evapotranspiration of the basin, the soil water volume content, the first parameter, and the second parameter. Where, the first parameter represents a systematic deviation of the estimated potential evapotranspiration, and the second parameter is configured to determine a curve shape of the actual evapotranspiration function.

The soil deep percolation function includes the functional relationship between the soil water volume content, the third parameter and the fourth parameter. Where, the third parameter represents the maximum value of the deep percolation, and the fourth parameter represents a water loss rate of the water-containing soil layer.

The basin runoff function includes the functional relationship between the input water flux function, the soil water volume content, and the fifth parameter. Where, the fifth parameter represents the runoff-forming rate of precipitation in the input water flux function.

The establishing module 604 is further configured to determine the sixth parameter according to the water quantity balance model. The sixth parameter represents the thickness of the researched soil layer of the basin.

In an embodiment, the runoff estimating device for the ungauged region further includes a testing module, configured to calculate the accuracy of the estimated monthly-scale runoff of the basin.

In an embodiment, the optimizing module 608 is further configured to calculate a gradient function of the objective function to the parameter.

The optimizing module 608 is configured to calculate a gradient value of the gradient function under the previous-cycle optimized value of the parameter.

The optimizing module 608 is configured to calculate a parameter value under the current-cycle gradient function, to obtain the current-cycle optimized value of the parameter.

In an embodiment, the optimizing module 608 is further configured to, according to the previous-cycle optimized value of the parameter, the gradient value and the preset optimization accuracy, perform an iterative computation on the parameter in the negative direction of the gradient to obtain the current-cycle optimized value of the parameter.

In an embodiment, a plurality of parameters to be calculated are configured and form a parameter space.

The optimizing module 608 is further configured to calculate the gradient functions of the objective function to the parameters in the parameter space, respectively.

The optimizing module 608 is configured to calculate gradient values of the gradient function under previous-cycle optimized values of the parameters, respectively.

The optimizing module 608 is further configured to, according to the previous-cycle optimized values of the parameters, the gradient values, and the optimization accuracy, perform iterative computations on the parameters in the negative direction of the gradient to obtain the current-cycle optimized values of the parameters. The optimized parameter values together constitute a current-cycle optimized parameter space.

For the specific limitations of the runoff estimating device for the ungauged region, please refer to the limitations of the runoff estimating method for the ungauged region above, and they will not be repeated here. Each module in the runoff estimating device for the ungauged region above may be implemented by software, hardware, and combinations thereof, either in whole or in part. The modules above may be embedded in or independent of the processor of the computer device in the form of hardware, or may be stored in the memory of the computer device in the form of software, so that the processor may call and execute operations corresponding to the modules above.

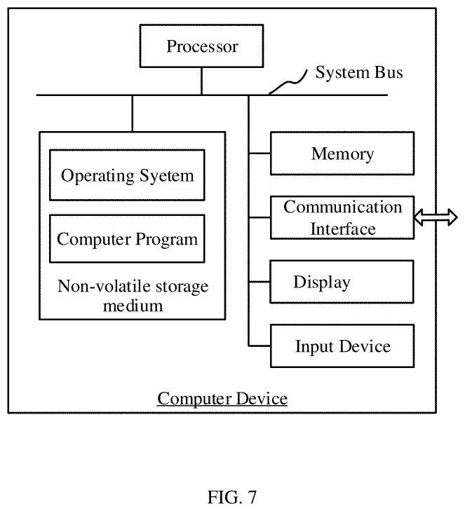

In an embodiment, a computer device is provided. The computer device may be a terminal, and the internal structure diagram may be as shown in FIG. 7. The computer device includes a processor, a memory, a communication interface, a display, and an input device, which are connected by a system bus. The processor of the computer device is configured to provide computing and control capabilities. The memory of the computer device includes non-volatile storage medium and internal memory. The non-volatile storage medium stores an operating system and computer programs. The internal memory provides an environment for the operation of the operating system and computer programs in the non-volatile storage medium. The communication interface of the computer device is used for wired or wireless communication with an external terminal, and the wireless communication may be realized by WIFI, operator network, NFC (Near Field Communication) or other technologies. When executed by the processor, the computer programs implement the runoff estimating method for the ungauged region. The display screen of the computer device may be a liquid crystal display screen or an electronic ink display screen, and the input device of the computer device may be a touch layer covering on the display screen, or a button, a trackball or a touch pad arranged on the shell of the computer device, or an external keyboard, a trackpad, or a mouse.

Those skilled in the art may understand that the structure shown in FIG. 7 is only a block diagram of partial structure related to the solution of the present application, and does not constitute a limitation on the computer device to which the solution of the present application is applied. The specific computer device may include more or fewer components than those shown in the figures, or may combine some components, or may have a different arrangement of components.

In an embodiment, a computer device is provided. The computer device includes a memory and a processor. Computer programs are stored on the memory, and when executing the computer programs, the processor performs the steps in the method of the embodiments above.

In an embodiment, a computer readable storage medium is provided, and computer programs are stored in the computer readable storage medium. When executed by the processor, the computer programs perform the steps in the method of the embodiments above.

Those ordinary skilled in the art may understand that all or part of the process in the method of the above-mentioned embodiments may be implemented by instructing the relevant hardware through a computer program, and the computer program may be stored in a non-volatile readable storage medium of the computer, when executed, the computer program may include the processes of the embodiments of the methods above. Wherein, any reference to memory, storage, database, or other media used in the various embodiments provided in this application may include at least one of non-volatile and volatile memory. Non-volatile memory may include read-only memory (Read-Only Memory, ROM), magnetic tape, floppy disk, flash memory, or optical memory, and the like. Volatile memory may include random access memory (RAM) or external cache memory. By way of illustration and not limitation, the RAM may be in various forms, such as static random-access memory (Static Random Access Memory, SRAM) or dynamic random-access memory (Dynamic Random Access Memory, DRAM).

The technical features involved in the embodiments above may be combined arbitrarily. For the sake of concision of the description, not all possible combinations of the technical features of the embodiments are described. However, as long as there is no contradiction in the combinations of these technical features, the combinations should be considered to be within the scope of the description.

What described above are several embodiments of the present invention, and these embodiments are specific and detailed, but not intended to limit the scope of the present application. It should be understood by the skilled in the art that various modifications and improvements may be made without departing from the scope of the present invention. Therefore, the scope of the present disclosure is defined by the appended claims.

What is claimed is:

1. A runoff estimating method for an ungauged region, comprising:
acquiring driving data, the driving data comprising monthly-scale precipitation, monthly-scale potential evapotranspiration, and soil water volume content of a basin within a preset period;
establishing an objective function of monthly-scale water quantity balance change of the basin, and determining a parameter to be calculated in the objective function;
initializing the parameter to obtain an initial value of the parameter;
inputting the initial value of the parameter and the driving data into the objective function to obtain an initial function value of the objective function;
performing an iterative computation on the parameter based on the initial value of the parameter and the initial function value to obtain an optimized value of the parameter, and inputting the optimized value of the parameter into the objective function to obtain an objective function value; and
when the objective function value meets preset conditions for an end of the iterative computation, estimating monthly-scale runoff of the basin within the preset period according to the optimized value of the parameter.

2. The method according to claim 1, wherein the performing an iterative computation on the parameter to obtain the optimized value of the parameter comprises:
calculating a gradient function of the objective function to the parameter;
calculating a gradient value of the gradient function under a previous-cycle optimized value of the parameter; and
calculating a current-cycle parameter value according to the gradient value to obtain a current-cycle optimized value of the parameter.

3. The method according to claim 2, wherein the calculating the current-cycle parameter value according to the gradient value to obtain the current-cycle optimized value of the parameter comprises:
performing the iterative computation on the parameter along a negative direction of the gradient according to the previous-cycle optimized value of the parameter, the gradient value, and a preset optimization accuracy, to obtain the current-cycle optimized value of the parameter.

4. The method according to claim 3, wherein:
a plurality of parameters to be calculated are configured and form a parameter space;
the calculating the gradient function of the objective function to the parameter comprises: calculating gradient functions of the objective function to the plurality of parameters in the parameter space, respectively;
the calculating the gradient value of the gradient function under the previous-cycle optimized value of the parameter comprises: calculating gradient values of the gradient function under previous-cycle optimized values of the plurality of parameters, respectively;
the performing the iterative computation on the parameter along the negative direction of the gradient according to the previous-cycle optimized value of the parameter, the gradient value, and the preset optimization accuracy, to obtain the current-cycle optimized value of the parameter, comprises: performing the iterative computations on the plurality of parameters along the negative direction of the gradient, according to the previous-cycle optimized values of the plurality of parameters, corresponding gradient values, and the optimization accuracy, to obtain the current-cycle optimized values of the plurality of parameters, and the current-cycle optimized values of the plurality of parameters constitute a current-cycle optimized parameter space;
the inputting the optimized value of the parameter into the objective function to obtain the objective function value comprises: inputting the current-cycle optimized values of the plurality of parameters into the objective function to obtain a current-cycle objective function value.

5. The method according to claim 1, wherein the establishing the objective function of monthly-scale water quantity balance change of the basin comprises:
converting a water flux change in the water quantity balance model to obtain a first soil water quantity change parameter;
obtaining a second soil water quantity change parameter according to a water quantity state change in the water quantity balance model;
establishing the objective function of the monthly-scale water quantity balance change of the basin according to the first soil water quantity change parameter and the second soil water quantity change parameter.

6. The method according to claim 5, wherein, a building process of the water quantity balance model comprises building the water quantity balance model according to a relationship between an input water flux function, an output water flux function, and the water quantity state within the preset period.

7. The method according to claim 6, wherein:
the parameter comprises a first parameter, a second parameter, a third parameter, a fourth parameter, a fifth parameter, and a sixth parameter;
an establishing process of the output water flux function comprises: obtaining the output water flux function by combining an actual evapotranspiration function, a soil deep percolation function, and a basin runoff function;
the actual evapotranspiration function comprises a functional relationship between the monthly-scale potential evapotranspiration of the basin, the soil water volume content, the first parameter, and the second parameter; the first parameter represents a systematic deviation of an estimated potential evapotranspiration, and the second parameter is configured to determine a curve shape of the actual evapotranspiration function;
the soil deep percolation function comprises a functional relationship between the soil water volume content, the third parameter, and the fourth parameter; the third parameter represents a maximum value of the deep percolation, and the fourth parameter represents a water loss rate of a water-containing soil layer;
the basin runoff function comprises a functional relationship between the input water flux function, the soil water volume content, and the fifth parameter; the fifth parameter represents a runoff-forming rate of precipitation in the input water flux function.

8. The method according to claim 7, wherein the output water flux function is:
L(θ)=ET(θ,a,b)+D(θ,c,d)+R(θ,f)
wherein ET(θ, a, b) represents the actual evapotranspiration function in mm/mon, and a, b represent the first parameter and the second parameter, represent the third parameter; D(θ, c, d) represents the soil deep percolation function in mm/mon, and c, d represent the third parameter and the fourth parameter, respectively; and R(θ, f) represents the basin runoff function in mm/mon, and f denotes the fifth parameter.

9. The method according to claim 8, wherein: the actual evapotranspiration function is
$$ET ( θ , a , b ) = \frac{PET ( t )}{2} \cdot [ 1 + tanh ( 8 \cdot [ \frac{θ}{φ} - sig ( b ) + 0.25 ] ) ] - a$$
wherein PET(t) represents the monthly-scale potential evapotranspiration of the basin in mm/mon; the first parameter a represents a systematic deviation of the estimated potential evapotranspiration;
$$sig ( b ) = \frac{1}{1 + {e}^{- b}} ;$$
φ represents a soil porosity degree;
the soil deep percolation function is
$$D ( θ , c , d ) = c \cdot {( \frac{θ}{φ} )}^{d} ,$$
wherein the third parameter c represents a maximum deep percolation, and the fourth parameter d represents a water loss speed of a water-containing soil layer; and
the basin runoff function is
$$R ( θ , f ) = P ( t ) \cdot {( \frac{θ}{φ} )}^{f} .$$

10. The method according to claim 9, wherein φ=(sand×0.395)+(clay×0.482)+(1−sand−clay)×0.451, wherein, sand and clay represent mass percentages of sand and clay in a total soil layer respectively.

11. The method according to claim 7, wherein: the water quantity balance model is
$$Δ z \frac{d θ}{dt} = P ( t ) - L ( θ ) ;$$
wherein Δz represents a thickness of a soil layer of the basin; θ represents the soil water volume content; P(t) represents the monthly-scale precipitation in mm/mon and is the input water flux of the basin; and L(θ) represents the output water flux in mm/mon.

12. The method according to claim 11, wherein:
the method further comprises determining the sixth parameter according to the water quantity balance model, wherein Δz denotes the sixth parameter.

13. The method according to claim 12, wherein Δz is a free parameter and is given an initial value of 300 cm.

14. The method according to claim 11, wherein:
the objective function of the monthly-scale water quantity balance change of the basin is
$$J = \sqrt{\frac{1}{N} {\sum }_{t = 1}^{N} {( \frac{d θ}{dt} - ( \frac{P ( t ) - L ( θ , X )}{Δ z} ) )}^{2}} ,$$
wherein N represents the number of samples, and X represents a parameter space of the parameters to be calculated, and
$$X = [ a , b , c , d , f , z ] ; \frac{P ( t ) - L ( θ , X )}{Δ z}$$
represents the first soil water quantity change parameter, and
$$\frac{d θ}{dt}$$
represents the second soil water quantity change parameter.

15. The method according to claim 1, wherein the preset conditions for the end of the iterative computations comprise: a difference between one objective function value obtained from a current-cycle optimization and another objective function value obtained from a previous-cycle optimization being less than an optimization threshold, and the optimization threshold being configured to be 1‰ of a magnitude of a soil water quantity change.

16. The method according to claim 2, wherein the current-cycle parameter value is calculated by
$${x}_{i + 1} = {x}_{i} - α \frac{\partial J}{\partial {x}_{i}} ,$$
wherein xᵢ₊₁ represents the current-cycle parameter value, xᵢ represents the previous-cycle optimized value of the parameter, and α represents an optimization accuracy.

17. The method according to claim 16, wherein α ranges from 1 to 10.

18. A runoff estimating device for an ungauged region, comprising:
an acquiring module, configured to acquire driving data, the driving data comprising monthly-scale precipitation, monthly-scale potential evapotranspiration, and soil water volume content of a basin within a preset period;
an establishing module, configured to establish an objective function of a monthly-scale water quantity balance change of the basin, and to determine a parameter to be calculated in the objective function;
an initializing module, configured to initialize the parameter to obtain an initial value of the parameter, and to input the initial value of the parameter and the driving data into the objective function to obtain an initial function value of the objective function;
an optimizing module, configured to perform an iterative computation on the parameter based on the initial value of the parameter and the initial function value, to obtain an optimized value of the parameter, and to input the optimized value of the parameter into the objective function to obtain an objective function value; and
a runoff calculating module, configured to, when the objective function value meets conditions for an end of the iterative computation, estimate a monthly-scale runoff of the basin within the preset period according to the optimized value of the parameter.

19. A computer device, comprising a memory and a processor, a computer program being stored on the memory, wherein, when performing the computer program, the processor executes steps of the method of claim 1.

20. A non-transitory computer readable medium, a computer program being stored on the non-transitory computer readable medium, wherein, when executed by a processor, the computer program executes steps of the method of claim 1.
